(19) United States
(12) Patent Application Publication
(10) Pub. No.: US 2012/0184591 A1
(43) Pub. Date: Jul. 19, 2012

(54) METHODS FOR TREATING PROSTATITIS

(75) Inventors: Gary HOEL, Murray, UT (US); Lawrence HILL, Farmington, UT (US); Kim CARAMELLI, Salt Lake City, UT (US)

(73) Assignee: WATSON PHARMACEUTICALS, INC., Corona, CA (US)

(21) Appl. No.: 13/033,483

(22) Filed: Feb. 23, 2011

Related U.S. Application Data
(60) Provisional application No. 61/432,571, filed on Jan. 13, 2011.

(51) Int. Cl. A61K 31/4045; A61P 29/00; A61P 15/16

(52) U.S. Cl. 514/415

(57) ABSTRACT

It has been discovered that silodosin is effective in treating patients having symptoms associated with prostatitis with silodosin or a pharmaceutically acceptable salt thereof. In a preferred embodiment, patients are treated with 4 mg once daily.

### CROSS-REFERENCE TO RELATED APPLICATIONS

This application claims the benefit of U.S. Provisional Patent Application Ser. No. 61/432,571, filed Jan. 13, 2011, which is incorporated herein by reference.

### FIELD OF THE INVENTION

The invention relates to compositions and methods for treating patients having symptoms associated with prostatitis with silodosin or a pharmaceutically acceptable salt thereof.

### BACKGROUND OF THE INVENTION

Prostatitis is the inflammation of the prostate. Four types of prostatitis are acute bacterial prostatitis, chronic bacterial prostatitis, asymptomatic inflammatory prostatitis, and chronic prostatitis/chronic pelvic pain syndrome (CP/CPPS).

CPPS is associated with genitourinary pain, pelvic pain and/or lower urinary tract syndrome (LUTS). LUTS include storage, voiding and postmicturition symptoms affecting the lower urinary tract.

Successful management of chronic prostatitis presents a difficult challenge for physicians and patients because of the lack of effective evidence-based, disease-specific treatment options. Prostatitis-like symptoms are relatively common in adult men, with an estimated prevalence in North America ranging from 2.2% to 9.7%. At least 90% of all cases of chronic prostatitis are attributable to abacterial chronic prostatitis/chronic pelvic pain syndrome (CP/CPPS).

CP/CPPS-associated symptoms can be debilitating. The National Institutes of Health has developed the Chronic Prostatitis Symptom Index (“CPSI”) as a way to measure the severity of symptoms associated with CP/CPPS. To calculate the CPSI, the patient answers questions regarding pain (scored 0-21), urinary symptoms (scored 0-10) and quality of life impact (scored 0-12). The lowest possible score is 0 (no symptoms); the highest possible score is 43 (worst symptoms). The Global Response Assessment (GRA) is a 7-question patient self-reported assessment that measures perception of change in symptoms (improvement, no change, or deterioration).

Of the available treatment options for CP/CPPS, none has consistently demonstrated efficacy in clinical studies. Results from clinical studies that evaluated the effects of α-blockers in patients with CP/CPPS have been inconclusive and contradictory. For example, in Nickel et al., “Alfuzosin and Symptoms of Chronic Prostatitis—Chronic Pelvic Pain Syndrome”, N Engl J. Med. 2008 Dec. 18; 359(25): 2663, the authors reported that the use of 10 mg once daily of alfuzosin was not more effective than placebo in reducing the symptoms of CP/CPPS.

Silodosin is an indoline derivative having the molecular formula C₂₅H₃₂F₃N₃O₄ and the following chemical structure:

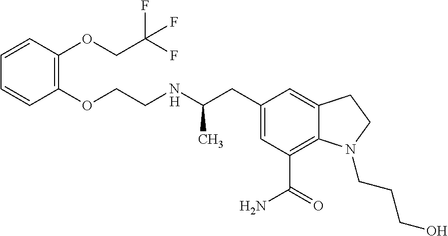

Silodosin is an α-adrenergic antagonist that has high selectivity for the α₁A receptor relative to α₁B and α₁D receptors. Silodosin is approved in Japan for 2 and 4 mg twice daily dosing to treat symptoms associated with benign prostatic hyperplasia (“BPH”). The synthesis of silodosin is described in U.S. Pat. No. 5,387,603, which is incorporated herein by reference.

### SUMMARY OF THE INVENTION

A preferred embodiment of the invention is a method of treating patients having symptoms of prostatitis and/or pelvic pain syndrome with silodosin.

### BRIEF DESCRIPTION OF THE DRAWINGS

FIG. 1 depicts the changing patient disposition over the course of the study described in Example 1.

FIG. 2 depicts the mean steady-state silodosin plasma concentration versus time profile in fed target-aged subjects after administering 8 mg silodosin QD (from Study SI06004).

FIG. 3 depicts the mean silodosin plasma concentration time profile by dose level and timepoint after multiple doses (from Study SI07004).

### DETAILED DESCRIPTION OF THE INVENTION

A patient having symptoms associated with CP/CPPS may be treated by administering a regimen of a pharmaceutical composition containing an effective amount of silodosin or a pharmaceutically acceptable salt thereof.

As used herein “effective amount” means an amount of silodosin or a pharmaceutically acceptable salt thereof that provides relief of CP/CPPS symptoms in a patient with CP/CPPS. Measures used to determine relief of CP/CPPS symptoms include, for example, CPSI. In preferred embodiments, the effective amount of silodosin is about 2-8 mg, about 4-8 mg, about 2 mg, about 4 mg, or about 8 mg.

As used herein “pharmaceutically acceptable salt” means those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describe pharmaceutically acceptable salts in detail in J. Pharmaceutical Sciences, 1977, 66: 1 et seq. The salts may be prepared in situ during the final isolation and purification of the compounds of the invention or separately by reacting a free base function with a suitable acid. Representative acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethanesulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which may be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid.

Basic addition salts can be prepared in situ during the final isolation and purification of compounds of this invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable basic addition salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like.

The pharmaceutical composition of silodosin may be administered in an immediate-release dosage form. Alternatively, the composition may be administered in an extended release dosage form. As used herein “immediate release” means any dosage form that is adapted to release about 50% or more, preferably about 60% or more, more preferably about 75% or more, of the active drug from the dosage form one hour from administration of said dosage form. As used herein “extended release” means any dosage form that is adapted to release more slowly than an immediate release dosage form. For example, an extended release dosage form may release over the course of 8 h, 12 h, or 24 h.

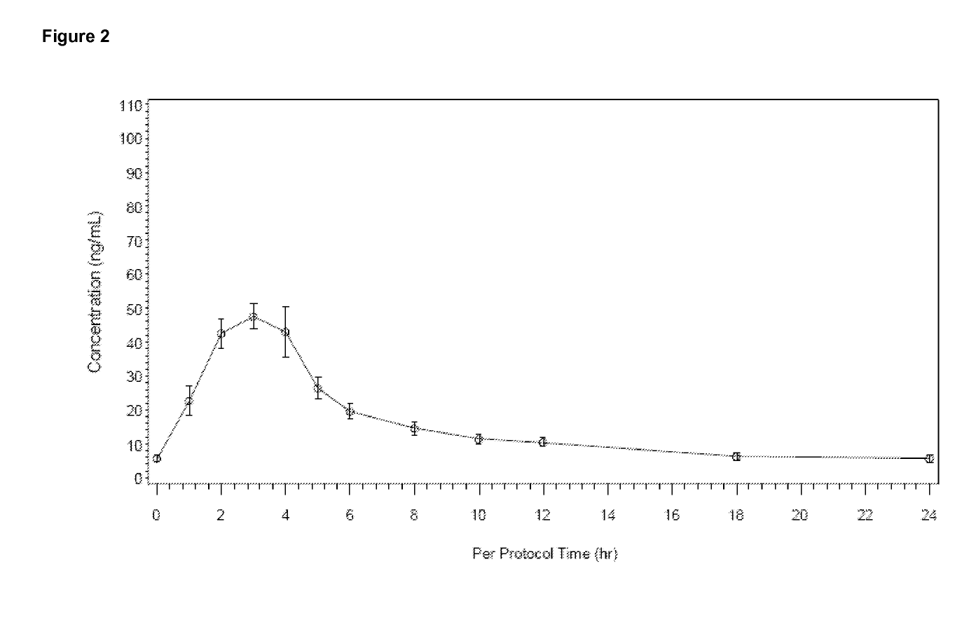

When an immediate-release 8 mg dosage form of silodosin was administered to patients once daily, the maximum blood plasma concentration of silodosin (Cmax) was reached at about 2.6 hours after administration. This is illustrated in Table 1 and FIG. 2. Study SI06004 assessed the area under the plasma concentration time curve for silodosin and four of its metabolites, as well as safety and tolerability, following administration of 8 mg once daily for 7 days.

TABLE 1
Pharmacokinetic Parameters of Silodosin
and Metabolites from Study SI06004

| | | compared to | | |
|---|---|---|---|---|
| | AUC(0-24), | silodosin | | |
| | ng * hr/mL | exposure | Cmax, ng/mL | Tmax, hr |
| silodosin | 373.4 (164.94) | 1.000 | 61.6 (27.54) | 2.6 (0.90) |
| 3213G | 1660.5 (647.23) | 4.447 | 102.4 (36.51) | 5.5 (2.29) |
| 3293 | 373.0 (141.72) | 0.999 | 34.3 (12.58) | 4.1 (1.29) |
| 3295 | 16.8 (19.30) | 0.045 | 3.4 (2.68) | 4.8 (2.62) |
| 3310 | 2.8 (3.86) | 0.007 | 1.6 (1.84) | 3.0 (1.12) |

Standard deviation is provided in parentheses.
The third column of the table compares AUC.
Half-lives were calculated for silodosin, KMD-3213G, and KMD-3293 as 13.3 ± 8.07, 24.1 ± 16.62, and 13.1 ± 7.10 hours, respectively.

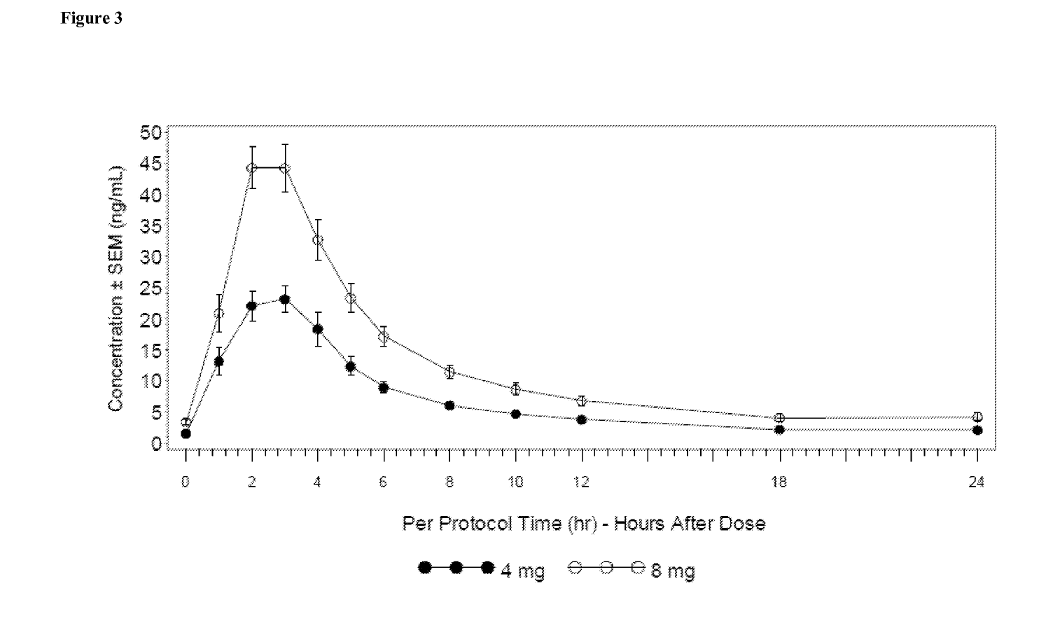

Silodosin pharmacokinetic parameters after 7 days of dosing are presented in Table 2, below. This data was obtained in Study SI07004, completed in October 2007, which investigated dose proportionality and safety of silodosin in healthy male subjects after one and seven daily doses of 4 mg or 8 mg. See also FIG. 3.

TABLE 2
Summary of silodosin pharmacokinetic parameters

| | | 4 mg | | 8 mg | |
|---|---|---|---|---|---|
| Parameter | Statistic | N = 22 | | N = 22 | |
| AUC [0-24] (ng · hr/mL) | Mean (SD) | 159.49 | (69.956) | 297.34 | (106.851) |
| | SEM | 14.915 | | 22.781 | |
| | CV (%) | 43.86 | | 35.94 | |
| | Median (25th, 75th percentile) | 156.16 | (115.18, 181.37) | 275.92 | (236.07, 361.66) |
| | Min, Max | 63.86, 375.04 | | 120.97, 594.14 | |
| | n | 22 | | 22 | |
| | P-value¹ | | | 0.5676 | |
| Cmax (ng/mL) | Mean (SD) | 28.36 | (12.401) | 51.14 | (17.144) |
| | SEM | 2.644 | | 3.655 | |
| | CV (%) | 43.73 | | 33.52 | |
| | Median (25th, 75th percentile) | 26.05 | (20.30, 32.30) | 50.30 | (40.50, 61.00) |
| | Min, Max | 9.85, 60.50 | | 16.20, 88.30 | |
| | n | 22 | | 22 | |
| | P-value¹ | | | 0.3909 | |
| Tmax (hr) | Mean (SD) | 2.4 | (0.73) | 2.5 | (0.80) |
| | SEM | 0.16 | | 0.17 | |
| | CV (%) | 30.5 | | 31.4 | |
| | Median (25th, 75th percentile) | 2.0 | (2.0, 3.0) | 2.0 | (2.0, 3.0) |
| | Min, Max | 1.0, 4.0 | | 2.0, 5.0 | |
| | n | 22 | | 22 | |
| kel (hr\hat{ }−1) | Mean (SD) | 0.0552 | (0.02419) | 0.0551 | (0.02086) |
| | SEM | 0.00555 | | 0.00455 | |
| | CV (%) | 43.8605 | | 37.8721 | |
| | Median (25th, 75th percentile) | 0.0489 | (0.0410, 0.0809) | 0.0573 | (0.0385, 0.0630) |
| | Min, Max | 0.0191, 0.0999 | | 0.0295, 0.0941 | |
| | n | 19 | | 21 | |
| t½ (hr) | Mean (SD) | 15.3 | (7.46) | 14.4 | (5.44) |
| | SEM | 1.71 | | 1.19 | |
| | CV (%) | 48.9 | | 37.6 | |
| | Median (25th, 75th percentile) | 14.2 | (8.6, 16.9) | 12.1 | (11.0, 18.0) |

| | | |
|---|---|---|
| Min, Max | 6.9, 36.3 | 7.4, 23.5 |
| n | 19 | 21 |

¹P-value is from the hypothesis test for dose proportionality; Dose proportionality claimed if P-value from T-test >0.05

A pharmaceutical composition of silodosin typically comprises silodosin or a pharmaceutically acceptable salt thereof and at least one pharmaceutically acceptable excipient. Pharmaceutically acceptable excipients include, for example, fillers, diluents, disintegrants, glidants, lubricants, and other excipients known in the art. Silodosin can also be co-administered with other active pharmaceutical ingredients, such as antibiotics. For example, for treating bacterial prostatitis, one embodiment of the invention is a composition comprising silodosin, an antibiotic and/or one or more excipients.

In one preferred embodiment, the pharmaceutical composition of silodosin comprises silodosin, a filler, a binder, a disintegrant and a lubricant. Optionally, the composition also contains a gelatin shell. In the following Table 1, the formulations used in Example 1 is provided.

TABLE 1
Silodosin Drug Product Composition

| Function | Component and Quality Reference |
|---|---|
| Drug Substance | Silodosin |
| Filler | D-Mannitol, USP |
| Binder | Pregelatinized Starch 1500, NF |
| Disintegrant | Pregelatinized Starch PCS, NF |
| Lubricant | Sodium Lauryl Sulfate, NF |
| Lubricant | Magnesium Stearate, NF |
| Granulating | Purified Water, USP* |
| Fluid | |
| Total | |

*Purified Water, USP is used for granulation, but is dried off during the manufacturing process and is not part of the final formulation.

While solid dosage forms are preferred, the dosage regimens of the invention are meant to include any dosage form, including liquid (e.g., a syrup), semi-solid (e.g., a gel), and transdermal patch dosage forms.

The pharmaceutical composition may be formulated into a solid dosage form by any method known to a person of ordinary skill in the art. Such methods include, but are not limited to, wet granulation, dry granulation by slugging and/or roller compaction, and direct compression. The solid dosage form may be in the form of a tablet (e.g., a compressed dosage form) or in the form of a capsule containing silodosin, optionally with one or more pharmaceutically acceptable excipients. The silodosin may be granulated, for example, with the pharmaceutically acceptable excipients.

Once-daily (QD) dosing regimens are generally preferred over divided dosing regimens, such as twice-daily (BID) or thrice-daily, because the former is generally more convenient and increases patient compliance. Accordingly, there is a need in the art for additional methods for treating the symptoms associated with CP/CPPS, particularly ones that include once-daily administration with silodosin.

Oral dosage forms can be administered with or without food. In Example 1, below, the capsules were administered with food.

Having described the invention with reference to certain preferred embodiments, other embodiments will become apparent to one skilled in the art from consideration of the specification. The invention is further defined by reference to the following examples. It will be apparent to those skilled in the art that many modifications, both to materials and methods, may be practiced without departing from the scope of the invention.

### Example 1

A 12-week, multicenter, double-blind, placebo-controlled, Phase II study (ClinicalTrials.gov identifier: NCT00740779) was conducted between September 2008 and October 2009. The protocol was approved by an Institutional Review Board before study initiation, and the study was conducted in accordance with the Declaration of Helsinki and the US Code of Federal Regulations. The primary end point was change from baseline to week 12 in the NIH—CPSI total score. Secondary end points included safety; change from baseline in NIH—CPSI pain, urinary, and HRQoL subscores; and change from baseline in SF-12 physical and mental component scores. In addition, responder analyses were conducted for the global response assessment (GRA) and NIH—CPSI at week 12. GRA responders were defined as subjects who indicated “markedly improved” or “moderately improved” on the 7-point GRA scale. NIH—CPSI responders were defined as subjects who had a decrease of 6 or more points in the NIH—CPSI total score.

Sample size calculation was based on published values for treatment- and placebo-associated changes from baseline in NIH—CPSI total. A difference of 5.2 between treatment groups was calculated as the detectable difference, if α=0.025, σ=7.3, power=0.9, and n=50 for each treatment group.

Participants

Study participants were recruited from 32 centers across the United States, including mostly community-based practices and a few university medical centers. Eligible participants were men aged 18 years or older with CP/CPPS who had a total NIH—CPSI score of at least 15 and an NIH—CPSI pain score of at least 8 at screening, had experienced pain in the pelvic region for at least 3 months before screening, and previously had not received α-blocker therapy for CP/CPPS. Patients were excluded if they had participated in a study of an investigational agent within the past 30 days, had experienced two or more urinary tract infections within the previous 12 months, or had medical conditions potentially precluding safe study participation or affecting study results, such as significant postural hypotension, abnormal test results of digital rectal examinations (except benign prostate enlargement), prostate-specific antigen >10.0 ng/mL, and liver or renal insufficiency. Patients treated with medications that might confound study results, such as α-blockers (for conditions other than CP/CPPS), 5α-reductase inhibitors, tricyclic antidepressants, androgens, and ketoconazole, had to undergo appropriate washout periods to be eligible.

Treatment and Assessment

Patients were randomized 1:1:1 to receive silodosin 4 mg, silodosin 8 mg, or placebo once daily with food at breakfast for 12 weeks. The formulation used in the study is provided in Table 1, above. Two doses of silodosin were chosen to evaluate the dose dependency of treatment effects in patients with CP/CPPS. The maximum dose of silodosin 8 mg/d was selected based on its established safety and efficacy profile in patients with BPH.

Baseline parameters were assessed after a 4-week screening period. All efficacy assessments were made with self-administered patient surveys. Patients completed the NIH—CPSI and subscales, GRA scale, and pain medication usage surveys at baseline and at weeks 4, 8, and 12 of the study. SF-12 was completed at baseline and at study end (week 12 or time of discontinuation). Safety was monitored over the course of the study through adverse events reporting, clinical laboratory tests, and vital signs assessments.

Statistical Analyses

All efficacy and safety data were summarized using descriptive statistics for continuous variables and frequency distributions for categorical variables. Efficacy end points were analyzed in the intent-to-treat (ITT) population, which consisted of all randomized patients with a baseline assessment for the NIH—CPSI total score. The primary efficacy variable and continuous secondary variables were analyzed for significance at α=0.05 (with Bonferroni correction for multiple comparisons) using analysis of covariance models with the baseline measurement of the efficacy variable as the covariate and effects of treatment. Because of the small number of patients in many participating centers, study center was not included in statistical analysis models. All analyses were conducted with SAS® v8.2 (SAS Institute Inc., Cary, N.C.). Last observation carried forward (LOCF) was used to impute missing values. For responder categorical variables, additional analyses were conducted based on observed cases and on the entire ITT population, with participants who did not complete the study being classified as nonresponders. GRA and NIH—CPSI responder analyses were done with chi-square tests at α=0.025 (two-sided) to account for multiple comparisons.

### Results

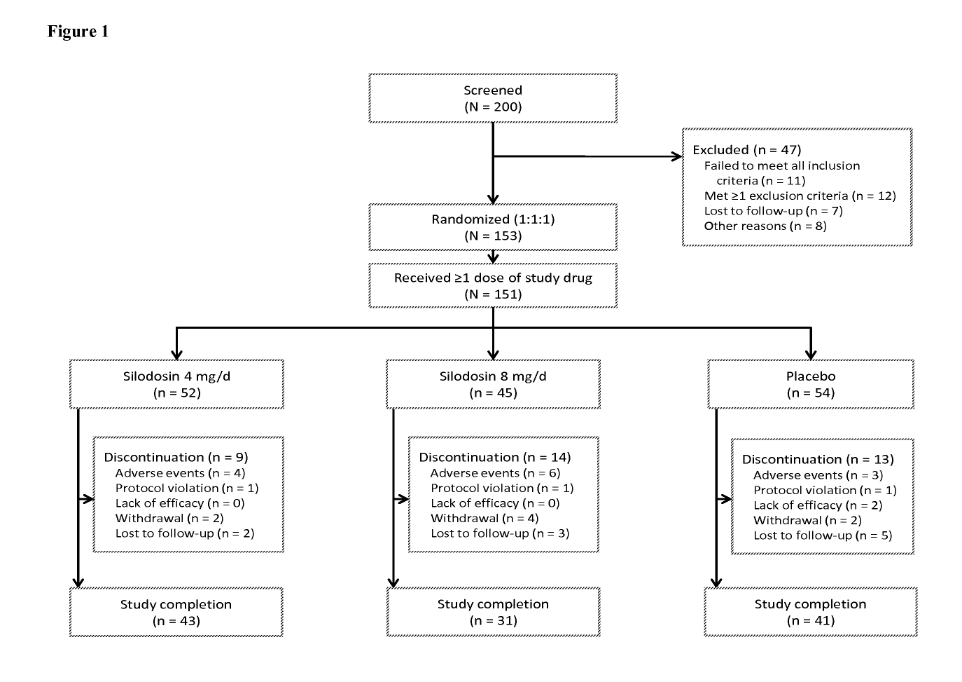

Of 200 patients screened, 153 were randomized (FIG. 1). Of those, two received no study medication and thus were excluded from the safety analysis. Of the 151 patients included in the safety analysis, 115 (76.2%) completed the study. Thirteen patients (8.6%) discontinued study participation because of adverse events, 10 (6.6%) were lost to follow-up, and eight (5.3%) withdrew voluntarily (FIG. 1). Demographic and baseline characteristics were similar for all three treatment groups (Tables 1 and 2). Median age was 48.2 years, and 80.1% of patients had experienced pain for at least 1 year.

Efficacy

Patients who received silodosin 4 mg experienced a significant decrease in total NIH—CPSI score (mean change±standard deviation, −12.1±9.3) compared with those who received placebo (−8.5±7.2; P=0.0224). Decreases in total NIH—CPSI scores also tended to be higher with silodosin 8 mg than placebo, but the differences were not statistically significant (Table 2). Further analyses of NIH—CPSI domain scores demonstrated statistically significant treatment effects of silodosin 4 mg versus placebo on the NIH—CPSI urinary symptom score (P=0.0102) and on the NIH—CPSI quality-of-life score (P=0.0099; Table 2). Silodosin 4 mg also provided increases in SF-12 physical component scores (4.2±8.1) that were significantly higher than those obtained with placebo (1.7±9.0; P=0.0492; Table 2).

Responder analyses for NIH—CPSI showed no statistically significant differences between treatment groups in the percentages of patients who achieved a reduction of at least 6 points in total score after 12 weeks (Table 3). Percentages of responders (with all early termination classified as nonresponder) were 54.9% for placebo, 59.6% for silodosin 4 mg (P=0.6287 versus placebo), and 46.7% for silodosin 8 mg (P=0.4205 versus placebo). In contrast, from week 4 to study end the percentage of patients who noted moderately or marked improvement on their GRA was significantly greater among patients treated with silodosin 4 mg than among those who received placebo (Table 3). If participants who discontinued were counted as nonresponders, responder rates for silodosin 4 mg and placebo were 55.8% and 29.4%, respectively (P=0.0069; Table 3).

Safety and Tolerability

Overall 51.7% of patients in this study experienced at least one adverse event; 33.1% experienced adverse events considered by the investigator to be related to a study drug. The most common drug-related adverse event was retrograde ejaculation (RE), which showed a dose-dependent incidence profile (Table 4). The percentage of patients with drug-related adverse events was greater in the silodosin 8 mg group than in the silodosin 4 mg group (Table 4). Except for RE, incidences of drug-related adverse events for silodosin 4 mg were similar to those for placebo (Table 4). Two patients who received silodosin 4 mg experienced serious adverse events, but these events were not considered drug-related by the investigator. One patient experienced severe urosepsis, which was resolved by treatment; the second patient experienced moderate syncope and consequently discontinued study participation.

The percentages of patients who discontinued study participation because of a drug-related adverse event were 13.3% for silodosin 8 mg, 5.8% for silodosin 4 mg, and 1.9% for placebo. Drug-related adverse events leading to discontinuation in the silodosin 8-mg group (n=6) were RE (n=4) and nasal congestion (n=2). Drug-related adverse events leading to discontinuation in the silodosin 4-mg group (n=3) were RE/libido decreased (n=1), ejaculation failure (n=1), and rash (n=1).

Beneficial treatment effects of silodosin 8 mg were observed and may prove to be statistically significant in a larger study.

TABLE 2
Demographic and baseline characteristics (safety population).

| | Silodosin 4 mg | | Silodosin 8 mg | | Placebo | |
|---|---|---|---|---|---|---|
| Characteristic | (n = 52) | | (n = 45) | | (n = 54) | |
| Age, years | | | | | | |
| Mean ± standard deviation | 49.2 | (13.3) | 46.7 | (15.6) | 49.0 | (11.6) |
| P value versus placebo* | 1.0000 | | 0.8229 | | | |
| Median (range) | 49.2 | (19.8 to 84.8) | 46.3 | (20.6 to 87.0) | 48.9 | (22.5 to 73.5) |
| Race, n (%) | | | | | | |
| White | 43 | (82.7) | 36 | (80.0) | 48 | (88.9) |
| Black | 7 | (13.5) | 6 | (13.3) | 4 | (7.4) |
| Asian | 2 | (3.8) | 1 | (2.2) | 1 | (1.9) |
| Other | 0 | | 2 | (4.4) | 1 | (1.9) |
| P value versus placebo^{†} | 0.4793 | | 0.7329 | | | |
| Ethnicity | | | | | | |
| Hispanic or Latino | 3 | (5.8) | 0 | | 3 | (5.6) |
| P value versus placebo^{‡} | 0.9620 | | 0.1083 | | | |
| Body mass index, n (%) | | | | | | |
| <32 kg/m² | 36 | (69.2) | 30 | (66.7) | 43 | (79.6) |
| ≧32 kg/m² | 16 | (30.8) | 15 | (33.3) | 11 | (20.4) |
| P value versus placebo^{‡} | 0.2193 | | 0.1445 | | | |
| Duration of pain symptoms, n | | | | | | |
| (%) | | | | | | |
| <1 year | 10 | (19.2) | 9 | (20.0) | 11 | (20.4) |
| ≧1 year | 42 | (80.8) | 36 | (80.0) | 43 | (79.6) |

| | | |
|---|---|---|
| P value versus placebo^{‡} | 0.8830 | 0.9635 |

*Analysis of variance.
^{†}Fisher's exact test.
^{‡}Chi-square test.

TABLE 3
Treatment effects on NIH-CPSI and SF-12 scores (ITT population)

| | Silodosin 4 mg | Silodosin 8 mg | Placebo |
|---|---|---|---|
| | (n = 52) | (n = 45) | (n = 51) |
| NIH-CPSI total score | | | |
| Baseline, mean ± SD | 26.0 ± 6.3 | 26.8 ± 5.9 | 27.9 ± 6.2 |
| Change from baseline, mean ± SD | (12.1 ± 9.3 | (10.2 ± 8.8 | (8.5 ± 7.2 |
| Adjusted mean difference versus | (4.2 ((7.9, (0.5) | (1.8 ((5.6, 2.0) | |
| placebo (95% CI) | | | |
| P value (versus placebo) | 0.0224 | 0.5912 | |
| NIH-CPSI pain score | | | |
| Baseline, mean ± SD | 12.3 ± 2.7 | 12.4 ± 3.2 | 12.8 ± 3.1 |
| Change from baseline, mean ± SD | −5.9 ± 4.5 | −4.9 ± 5.0 | −4.5 ± 3.6 |
| Adjusted mean difference versus | −1.6 (−3.4, 0.3) | −0.5 (−2.4, 1.4) | |
| placebo (95% CI) | | | |
| P value (versus placebo) | 0.1229 | 1.0000 | |
| NIH-CPSI quality-of-life impact | | | |
| score | | | |
| Baseline, mean ± SD | 8.3 ± 2.1 | 8.7 ± 2.2 | 8.6 ± 2.4 |
| Change from baseline, mean ± SD | −4.1 ± 3.1 | −3.5 ± 3.0 | −2.7 ± 2.5 |
| Adjusted mean difference versus | −1.5 (−2.8, −0.3) | −0.7 (−2.0, 0.5) | |
| placebo (95% CI) | | | |
| P value (versus placebo) | 0.0099 | 0.3783 | |
| NIH-CPSI urinary symptoms score | | | |
| Baseline, mean ± SD | 5.3 ± 3.0 | 5.7 ± 2.5 | 6.5 ± 2.6 |
| Change from baseline, mean ± SD | −2.2 ± 2.7 | −1.8 ± 2.4 | −1.3 ± 3.0 |
| Adjusted mean difference versus | −1.4 (−2.5, −0.3) | −0.8 (−1.9, 0.3) | |
| placebo (95% CI) | | | |
| P value (versus placebo) | 0.0102 | 0.2115 | |
| SF-12 physical component | | | |
| Baseline, mean ± SD | 46.4 ± 9.7 | 45.3 ± 10.0 | 45.3 ± 10.8 |
| Change from baseline, mean ± SD | 4.2 ± 8.1 | 4.4 ± 7.0 | 1.7 ± 9.0 |
| Adjusted mean difference versus | 3.0 (0.01, 6.0) | 2.7 (−0.4, 5.8) | |
| placebo (95% CI) | | | |
| P value (versus placebo) | 0.0492 | 0.0976 | |
| SF-12 mental component | | | |
| Baseline, mean ± SD | 46.8 ± 9.3 | 46.3 ± 10.3 | 43.3 ± 11.5 |
| Change from baseline, mean ± SD | 3.2 ± 7.3 | 0.5 ± 7.2 | 2.9 ± 10.0 |
| Adjusted mean difference versus | 1.5 (−1.9, 4.9) | −1.4 (−4.9, 2.1) | |
| placebo (95% CI) | | | |
| P value (versus placebo) | 0.6167 | 0.7177 | |

CI = confidence interval;
SD = standard deviation.

TABLE 4
Response rates for NIH-CPSI and GRA

| | Silodosin 4 mg | Silodosin 8 mg | Placebo |
|---|---|---|---|
| Patients, n (%) | (n = 52) | (n = 45) | (n = 51) |
| NIH-CPSI responders | | | |
| Week 4 OC | 36 (81.8)* | 29 (87.9)* | 25 (56.8) |
| Week 8 OC | 33 (82.5) | 26 (89.7) | 28 (70.0) |
| Week 12 OC | 31 (73.8) | 21 (77.8) | 28 (71.8) |
| Week 12 LOCF | 36 (69.2) | 31 (68.9) | 31 (60.8) |
| Week 12 ET | 31 (59.6) | 21 (46.7) | 28 (54.9) |
| GRA responders | | | |
| Week 4 OC | 24 (52.2)* | 15 (42.9) | 11 (24.4) |
| Week 8 OC | 25 (58.1)* | 15 (46.9) | 11 (26.8) |
| Week 12 OC | 29 (67.4)* | 15 (51.7) | 15 (38.5) |
| Week 12 LOCF | 32 (62.7)* | 21 (51.2) | 17 (34.7) |
| Week 12 ET | 29 (55.8)* | 15 (33.3) | 15 (29.4) |

*P < 0.025 versus placebo.
NIH-CPSI responders had a reduction of ≧6 points in total score.
GRA responders indicated that their condition had moderately or markedly improved upon treatment initiation (6 or 7 points on the 7-point GRA scale).
ET = early termination counted as nonresponse
OC = observed cases
LOCF = last observation carried forward

TABLE 5
Drug-related adverse events in ≧2% of the safety population

| | Silodosin 4 mg | Silodosin 8 mg | Placebo |
|---|---|---|---|
| Patients, n (%) | (n = 52) | (n = 45) | (n = 54) |
| Retrograde ejaculation | 14 (26.9) | 18 (40.0) | 1 (1.9) |
| Dizziness | 1 (1.9) | 4 (8.9) | 4 (7.4) |
| Headache | 0 | 2 (4.4) | 1 (1.9) |
| Nasal congestion | 2 (3.8) | 3 (6.7) | 0 |
| Libido decreased | 1 (1.9) | 1 (2.2) | 1 (1.9) |

What is claimed is:

1. A method of treating a patient having symptoms of prostatitis and/or pelvic pain syndrome comprising administering to said patient an effective amount of silodosin or a pharmaceutically acceptable salt thereof.

2. The method of claim 1, wherein the effective amount is about 2 mg to about 8 mg.

3. The method of claim 1, wherein the effective amount is about 4 mg to about 8 mg.

4. The method of claim 1, wherein the effective amount is 4 mg to 8 mg.

5. The method of claim 1, wherein the effective amount is about 4 mg.

6. The method of claim 1, wherein the effective amount is delivered in a divided daily dose.

7. The method of claim 1, wherein the effective amount is delivered in a single daily dose.

8. The method of claim 5, wherein the effective amount is delivered in a twice-daily dose.

9. The method of claim 5, wherein the effective amount is delivered in a single daily dose.

10. The method of claim 1, wherein a pharmaceutical composition is administered comprising silodosin and at least one excipient.

11. The method of claim 1, wherein a pharmaceutical composition is administered comprising silodosin and an antibiotic.

12. A method of treating a patient having symptoms of chronic prostatitis/chronic pelvic pain syndrome comprising administering to said patient an effective amount of silodosin or a pharmaceutically acceptable salt thereof.

13. The method of claim 12, wherein the effective amount is about 2 mg to about 8 mg.

14. The method of claim 12, wherein the effective amount is about 4 mg.
